(19) United States
(12) Patent Application Publication
(10) Pub. No.: US 2024/0398574 A1
(43) Pub. Date: Dec. 5, 2024

(54) PROSTHESIS, ESPECIALLY KNEE PROSTHESIS OR HIP STEM PROSTHESIS

(71) Applicant: Aesculap AG, Tuttlingen (DE)

(72) Inventor: Harry Kempf, Stetten am kalten Markt (DE)

(21) Appl. No.: 18/660,378

(22) Filed: May 10, 2024

(30) Foreign Application Priority Data
Jun. 2, 2023 (DE) 10 2023 114 598.3

(51) Int. Cl. A61F 2/30

(52) U.S. Cl. CPC A61F 2/30771; A61F 2002/30322; A61F 2002/30594; A61F 2002/30878

(57) ABSTRACT

A prosthesis, in particular a knee prosthesis or hip stem prosthesis, includes a shaft for arrangement and mounting in a long bone. The shaft has shaft sections extending parallel to the shaft axis that are separated from one another by a slotted intermediate space. The shaft portions are deformable by bending loads starting from an undeformed resting state such that the slotted intermediate space decreases. A first shaft portion includes a first projection provided on a side of the first shaft portion facing a second shaft portion and that forms a first constriction of the intermediate space. The first shaft portion also includes a first boundary surface for alignment against the second shaft portion. The first boundary surface is spaced at a distance from the second shaft portion in the undeformed resting state.

### CROSS-REFERENCE TO RELATED APPLICATION(S)

This application claims priority under 35 U.S.C. § 119 to German Application No. 10 2023 114 598.3, filed on Jun. 2, 2023, the content of which is incorporated by reference herein in its entirety.

### FIELD

The present disclosure relates to a prosthesis, in particular a knee prosthesis or hip stem prosthesis, having a shaft for arrangement and mounting in a long bone, wherein the shaft extends lengthwise along a shaft axis between a proximal end and a distal end that can be inserted into the long bone, wherein the shaft has a first, in particular solid, cross-section originating from the proximal end along a first partial length, and wherein the shaft has a second cross-section deviating from the first cross-section along a second partial length that includes the distal end of the shaft, wherein the shaft comprises shaft portions extending parallel to the shaft axis along the second partial length, and said shaft portions are separated from one another by a slotted intermediate space, wherein the shaft portions-starting from a not deformed resting state—are deformable by a bending load such that the slotted intermediate space decreases.

### BACKGROUND

Prostheses of the aforementioned type are for example known from EP 0 966 928 A2. To fasten the shaft in a long bone, the distal end of the shaft is inserted into a bone marrow space of the long bone. The purpose of the slotted intermediate space is to allow the shaft portions to deform elastically and radially inward during their insertion into the bone marrow space, wherein the slotted intermediate space decreases. After complete insertion into the bone marrow space, the shaft sections rest under pretension against a boundary of the bone marrow space.

It has been shown that so-called shaft pain can also occur as a result of this pretensioning, at least in some patients, even for an extended time after the prosthesis is inserted. It is therefore necessary to provide shaft portions that are deformable with low forces in order to reduce the elastic restoration forces in this way. However, the problem presented at the same time is that an overly unstable adaptation of the shaft portions could be accompanied by an undesirable plastic deformation or even a failure of the shaft portions.

### SUMMARY

Based on this, the underlying object of the present disclosure is to provide a prosthesis that develops the lowest possible restoration forces but at the same time has sufficiently high stability.

This object is achieved according to the present disclosure by a prosthesis of the type mentioned above in that a first shaft portion comprises a first projection that is provided on a side of the first shaft portion facing a second shaft portion, forms a first constriction of the intermediate space and is arranged at a distance from the first partial length of the shaft, as seen along the shaft axis, wherein the first projection comprises a first boundary surface for alignment against the second shaft portion, wherein the first boundary surface is spaced at a distance from the second shaft portion in the not deformed resting state.

The prosthesis according to the present disclosure specifies at least one first shaft portion whose maximum bending deformation is limited by a projection having a first boundary surface that is aligned against the second shaft portion in a maximum deformed state of the first shaft portion. In a not deformed resting state, the cross-section of the slotted intermediate space is constricted in the region of the first projection. In this not deformed resting state of the first shaft portion, the first boundary surface of the projection is spaced at a distance from the second shaft portion. This allows the first shaft portion to also deform at the height of the first projection—as seen along the shaft axis—when a bending load is exerted. However, the deformation path of the first shaft portion at the height of the first projection is limited to a state wherein the first boundary surface of the first projection of the first shaft portion is aligned against the second shaft portion.

It is in particular possible that the first shaft portion has a comparatively small surface moment of inertia, i.e., can be deformed with only low deformation forces when the prosthesis is surgically inserted into the long bone and develops comparatively low restoration forces after insertion into the bone marrow space. An undesired plastic deformation or even a failure with breakage of the first shaft portion can nevertheless be prevented because the first shaft portion cannot further deform as a result of the alignment of the first boundary surface against the second shaft portion. The maximum deformation path of the first shaft portion is limited to the extent by which the projection constricts the slotted intermediate space. This extent is defined by the height of the first projection, which extends beyond a base plane of the first shaft portion extending in the direction toward the second shaft portion.

The inventive prosthesis allows the use of materials that have proven themselves in practice, such as titanium or steel alloys.

Special advantages result when the second shaft portion has a second projection that is provided on a side of the second shaft portion facing the first shaft portion, forms a second constriction of the intermediate space, and is arranged at a distance from the first partial length of the shaft, as seen along the shaft axis, wherein the second projection comprises a second boundary surface for alignment against the first shaft portion, wherein the second boundary surface is spaced at a distance from the first shaft portion in the not deformed resting state.

The first shaft portion and the second shaft portion are preferably arranged on opposite sides of a central shaft axis. When the second shaft portion is also provided with a second projection, the deformation path of the second shaft portion can also be limited when the second boundary surface of the second projection is aligned against the first shaft portion.

It is possible for the first projection and the second projection to be arranged at an offset from one another along the shaft axis such that one of the two boundary surfaces is aligned against one of the two shaft sections when a bending load is applied, and such that the other boundary surface is only subsequently aligned against the other shaft section.

However, it is also possible that the first projection and the second projection are arranged along the shaft axis at the same height—as seen along the shaft axis—and that a bending deformation of the shaft sections in the region of the first projection and the second projection is limited by an alignment of the first boundary surface against the second boundary surface. This is particularly advantageous when the shaft sections have mirror-symmetrical cross-sections in relation to one another relative to the shaft axis, with the consequence that the two shaft sections deform symmetrically relative to the shaft axis when the distal end is inserted into a long bone and exert restoration forces opposite and equal in value on opposing sections of a boundary of the bone marrow space when the prosthesis is in the inserted state.

It is further preferred when the first boundary surface and the second boundary surface are oriented parallel to one another in the not deformed resting state of the shaft portions or in a deformed state of the shaft portions. In the case of a parallel orientation of the boundary surfaces in the not deformed resting state of the shaft portions and a deformation of the shaft portions, the two boundary surfaces initially only come into contact with one another along a contact line. In the case of further deformation, this contact line expands to a state wherein the boundary surfaces are aligned against each other (i.e., over a surface area) along their extent. In this state, the two shaft portions can be braced against one another in a stable manner, namely by avoiding locally increased surface pressures. This applies in particular when the boundary surfaces are arranged at a slight angle in relation to one another in a not deformed resting state, wherein the angle is selected such that the two boundary surfaces are aligned against each other simultaneously and along their entire extent in the maximum deformed state of the shaft sections.

It is particularly preferred that the first boundary surface and/or the second boundary surface extend along the shaft axis up to the distal end of the shaft. As a result, a deformation path of a shaft portion or the shaft portions can be limited in a region of the shaft portions that has a maximum deformation path during a bending load. It is also advantageous that the slotted intermediate space on the distal end of the shaft has a cross-section constricted as a result of the projections, thus allowing the distal end to be inserted particularly easily into a bone marrow space.

The present disclosure further proposes that the first boundary surface and/or the second boundary surface have a boundary surface lengthwise along the shaft axis that is between 5% and 45%, in particular between 15% and 35%, of the second partial length. In other words: 95% to 55%, in particular 85% to 65%, of the second partial length of the shaft sections preferably has no projections, i.e., has a slotted intermediate space that is not constricted by means of at least one projection. This “projection-free” second partial length of the shaft sections allows the provision of a large-volume intermediate space, which is accompanied by a corresponding reduction in the cross-section of the shaft sections, which thus have low deformation resistances.

In a region of the second partial length of the shaft adjoining the first partial length of the shaft and in the not deformed resting state of the shaft portions, the shaft portions comprise a shaft portion distance corresponding to the width of the slotted intermediate space. This (“projection-free”) shaft portion distance is preferably at least 20%, in particular at least 25%, of a maximum diameter of a circumferential surface of the shaft. For example, this maximum diameter is 10 mm, 12 mm, 14 mm, 16 mm, 18 mm, 20 mm, 22 mm, or 24 mm. For the example of a shaft with a diameter of 10 mm, it is therefore preferred that the aforementioned shaft portion distance is at least 2 mm, such that the radial extent of the shaft portions is a maximum of 4 mm, given a symmetrical design relative to the shaft axis.

The first projection has a first height measured perpendicular to the first boundary surface, and by which the first projection extends beyond a base plane of the first shaft portion. If present, the second projection has a second height measured perpendicular to the second boundary surface, and by which the second projection extends beyond a base plane of the second shaft portion. The aforementioned base planes form opposing boundary surfaces of the slotted intermediate space, as seen along the shaft axis in a region in which no projection is arranged. The present disclosure proposes that the first height and/or the second height is or are at least 0.5 mm. This means that the slotted intermediate space is constricted by a deformation path of at least 0.5 mm in the region of at least one projection, as compared to a prosthesis known from the prior art that only has opposing base planes and no inventive projection or inventive projections.

When two projections are provided that are arranged along the shaft axis at the same height, the slotted intermediate space is constricted by at least 1 mm compared to the prior art, and a common deformation path of the shaft portion is thus shortened by at least 1 mm compared to the prior art.

It is in particular preferred when a ratio between the first elevation or the second elevation, or between the sum of the first height or the second height, or between the sum of the first height and the second height on the one hand and the (“projection-free”) shaft section distance on the other, is at least 10%, in particular at least 20%. This is accompanied by a shortening of the deformation path—in reference to the “projection-free” prior art—by at least 10%, in particular by at least 20%.

It is further advantageous when the shaft has a circumferential surface with longitudinal grooves extending parallel to the shaft axis. Such a design of a circumferentially closed surface of a shaft can also be described in that the shaft has a “Wagner profile”. The longitudinal grooves support a reliable anchoring at a boundary of the bone marrow space, but—when taken by themselves—form locally weakened areas of the cross-section of the shaft sections. The prosthesis according to the present disclosure allows compensation for this weakening.

### BRIEF DESCRIPTION OF THE DRAWINGS

Further features and advantages of the present disclosure are the scope of the following description of a preferred exemplary embodiment.

FIG. 1 shows a sectional view of a prosthesis known from the prior art;

FIG. 2 shows a side view of an embodiment of a prosthesis according to the present disclosure; and

FIG. 3 shows an enlarged view of a section labeled as III in FIG. 2.

### DETAILED DESCRIPTION

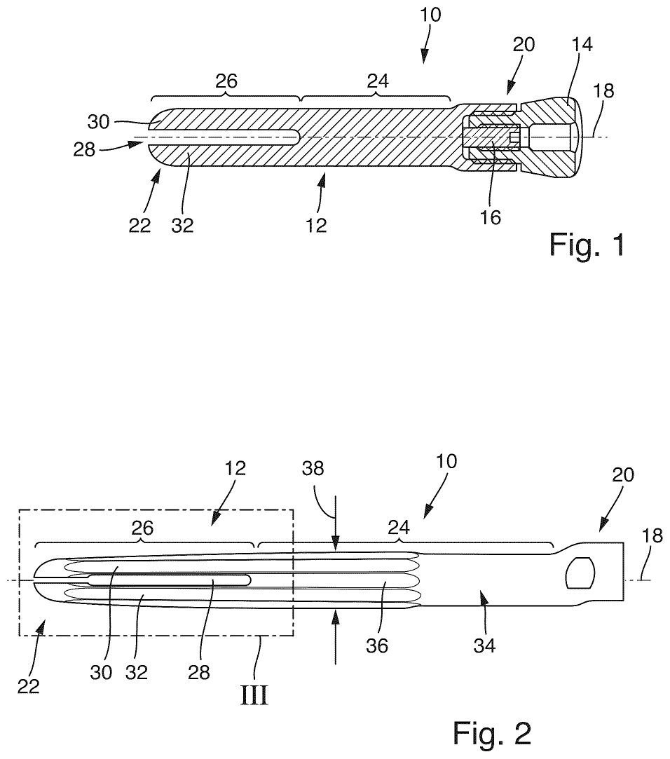

A prosthesis known from the prior art (EP 0 966 928 A2) is shown in FIG. 1 and is labeled with the reference symbol 10. The prosthesis comprises a shaft 12 that is connected to a joint section 14 at one end, for example by positioning a connecting part 16 in between them.

The shaft 12 extends along a shaft axis 18, namely between a proximal end facing the joint section 14 and a free distal end 22 that is inserted first into the bone marrow space when the shaft is arranged in a bone marrow space of a long bone.

Starting from the proximal end 20, the shaft 12 has a first, in particular solid, cross-section along a first partial length 24. Along a second partial length 26, which includes the distal end 22 of the shaft 12, the shaft 12 has a second cross-section that deviates from the first cross-section.

A slotted intermediate space 28 extending parallel to the shaft axis 18 is provided along the second partial length 26. The intermediate space 28 is delimited on opposing sides by a first shaft portion 30 and a second shaft portion 32 spaced at a distance from the first shaft portion 30.

Reference is made to the above description of FIG. 1 for the following description of an exemplary embodiment of an inventive prosthesis 10. The prosthesis 10 according to FIG. 2 can for example be adapted as a knee prosthesis or hip stem prosthesis; it goes without saying that a joint portion 14 (not shown) can also be provided in the prosthesis 10, if necessary by using an intermediate part 16.

The shaft 12 comprises a circumferential surface 34, which—as seen along the shaft axis 18—is provided at least sectionally with longitudinal grooves 36 that are distributed, in particular distributed regularly, over the circumference of the shaft 12. For example, a total of ten longitudinal grooves 36 are provided at an offset in relation to one another in circumferential direction.

The shaft 12 has a maximum diameter 38, for example between 10 mm and 24 mm.

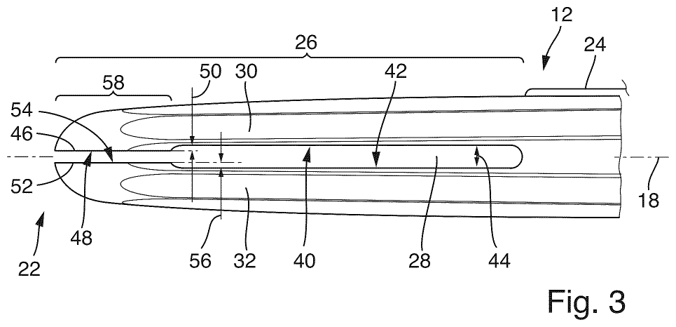

Reference is made to FIG. 3 for the further description.

The first shaft portion 30 comprises a base plane 40 facing the second shaft portion 32 in a region adjoining the first partial length 24 of the shaft 12. The second shaft portion 32 comprises a second base plane 42 facing the first base plane 40. The base planes 40 and 42 are spaced at a distance from one another over a shaft portion distance 44 and form boundaries of the slotted intermediate space 28.

On its side facing the second shaft portion 32, the first shaft portion 30 has a projection 46 that comprises a first boundary surface 48 facing the second shaft portion 32. The distance between the first boundary surface 48 and the base plane 40 corresponds to a height 50 of the first projection 46. The first projection 46 is in particular formed integrally with the first shaft portion 30.

On its side facing the first shaft portion 30, the second shaft portion 32 carries a second projection 52 with a second boundary surface 54 facing the first shaft portion 30. The distance of the second boundary surface 54 to the base plane 42 of the second shaft portion 32 corresponds to a second height 56 of the second projection 52. The second projection 52 is in particular formed integrally with the first shaft portion 30.

The first height 50 is at least 0.5 mm; the second height 56 is at least 0.5 mm.

The projections 46 and 52 form respective constrictions of the slotted intermediate space 28. In the region of the boundary surfaces 46, 48, the slotted intermediate space 28 is constricted by the sum of the heights 50 and 56 of the projections 46 and 52.

The boundary surfaces 48 and 54 are oriented parallel to one another, in particular in a not deformed resting state of the shaft 12.

The boundary surfaces 48 and 54 are arranged relative to the shaft axis 18 on the sides of the shaft axis 18 facing away from one another and are mirror-symmetrical with respect to their dimensions and position in relation to the shaft axis 18.

The boundary surfaces 48 and 54 extend along the shaft axis 18 over a preferably identical boundary surface length 58.

Starting from the not deformed resting state of the shaft sections 30 and 32 shown in the drawing, the distal end 22 can be inserted into a bone marrow space of a long bone, in particular a tibia or a femur. In this case, the diameter 38 of the shaft 12 is selected in relation to the diameter of the bone marrow space such that the shaft can preferably be inserted into the bone marrow space with the extension of the longitudinal grooves 36, and such that the surfaces of the shaft sections 30 and 32 pointing radially outward are after said insertion aligned under pretension against the respective boundary sections of the bone marrow space.

Starting from a not deformed starting state, see FIG. 3, the shaft portions 30 and 32 can be deformed by bending, wherein the boundary surfaces 48 and 54 move toward each other, namely up to a state wherein the boundary surfaces 48 and 54 are aligned against each other at least sectionally, preferably over a surface area. This alignment defines a maximum deformation path of the shaft sections 30 and 32 across the boundary surface length 58.

Assuming a shaft section distance 44 identical to the prior art (see FIG. 1) and assuming identical materials and identical dimensioning of the shaft sections 30 and 32 and assuming an identical second partial length 26, the deformation path of the shaft sections 30 and 32 is thus shortened in the region of the distal end 28 by a measure that corresponds to the sum of the heights 50 and 56 of the projections 46 and 52. The resulting effect is that the shaft sections 30 and 32 can be bent with low deformation forces and, accordingly, only develop low restoration forces after insertion into a long bone, while also preventing an undesired plastic deformation or failure with breakage of the shaft sections 30 and 32.

1. A prosthesis comprising a shaft for arrangement and mounting in a long bone, wherein the shaft extends along a shaft axis between a proximal end and a distal end that is insertable into the long bone, wherein, starting from the proximal end, the shaft has a solid cross-section along a first partial length and wherein the shaft has a second cross-section deviating from the first cross-section along a second partial length, said second cross-section including the distal end of the shaft, wherein the shaft has shaft portions extending along the second partial length parallel to the shaft axis that are separated from one another by a slotted intermediate space, wherein the shaft portions are deformable—starting from an undeformed resting state—by bending loads such that the slotted intermediate space decreases, wherein a first shaft portion comprises a first projection on a side of the first shaft portion facing a second shaft portion and forms a first constriction of the intermediate space and is arranged at a distance from the first partial length of the shaft, as seen along the shaft axis, wherein the first projection comprises a first boundary surface for alignment against the second shaft portion, wherein the first boundary surface is spaced at a distance from the second shaft portion in the undeformed resting state.

2. The prosthesis according to claim 1, wherein the second shaft portion comprises a second projection on a side of the second shaft portion facing the first shaft portion and forms a second constriction of the intermediate space and is arranged at a distance from the first partial length of the shaft, as seen along the shaft axis, wherein the second projection comprises a second boundary surface for alignment against the first shaft portion, wherein the second boundary surface is spaced at a distance from the first shaft portion in the undeformed resting state.

3. The prosthesis according to claim 2, wherein a bending deformation of the first shaft portion and the second shaft portion is limited in a region of the first projection and the second projection by an alignment of the first boundary surface against the second boundary surface.

4. The prosthesis according to claim 3, wherein the first boundary surface and the second boundary surface are oriented parallel to one another in the undeformed resting state of the first shaft portion and the second shaft portion or in a deformed state of the first shaft portion and the second shaft portion.

5. The prosthesis according to claim 1, wherein the first boundary surface and/or the second boundary surface extends or extend up to the distal end of the shaft, as seen along the shaft axis.

6. The prosthesis according to claim 1, wherein the first boundary surface and/or the second boundary surface has or have a boundary surface length between 5% and 45% of the second partial length, as seen along the shaft axis.

7. The prosthesis according to claim 1, wherein the shaft portions—in a region of the second partial length of the shaft adjoining the first partial length of the shaft and in the undeformed resting state of the shaft portions—have a shaft portion distance from one another corresponding to a width of the slotted intermediate space.

8. The prosthesis according to claim 7, wherein the first projection has a first height measured perpendicularly to the first boundary surface and/or in that the second projection has a second height measured perpendicularly to the second boundary surface.

9. The prosthesis according to claim 8, wherein a ratio between the first height or the second height or between a sum of the first height and the second height and the shaft portion distance is at least 10%.

10. The prosthesis according to claim 1, wherein the shaft comprises a circumferential surface with longitudinal grooves extending parallel to the shaft axis.
